(19) United States
(12) Patent Application Publication
(10) Pub. No.: US 2013/0072791 A1
(43) Pub. Date: Mar. 21, 2013

(54) Contrast saver for invasive angiographic procedures

(71) Applicants: Philip Paspa, Hickory, NC (US); Paul Paspa, Los Gatos, CA (US)

(72) Inventors: Philip Paspa, Hickory, NC (US); Paul Paspa, Los Gatos, CA (US)

(21) Appl. No.: 13/137,825

(22) Filed: Sep. 15, 2011

(51) Int. Cl. A61B 6/00

(52) U.S. Cl. 600/431

(57) ABSTRACT

Herein is described a device that makes it possible to use opened but unused contrast material safely for subsequent patients. During invasive angiographic procedures, fixed contrast bottles are used. These have fixed amounts of contrast in them, and very often when the bottles are not used completely, the excess goes to waste. The present device allows the unused portion to be used with the next patient. By using a large source of contrast, such as a bottle, with a docking unit, controlled amounts of contrast may be added to a patient line and then the extra contrast may be used for the next patient, with the use of a fresh receiver. The docking unit comprises a dosing unit and a receiver.

### BACKGROUND OF THE INVENTION

The present invention generally relates to liquid dispensing systems, and, more specifically, to a medical device that makes it possible to use unused contrast material from opened containers safely for subsequent patients.

There is a great deal of wasted contrast material that is unused during invasive angiographic procedures, such as cardiac catheterization. During invasive angiographic procedures, fixed volume contrast bottles are used. These have fixed amounts of contrast in them, and very often when the bottles are not used completely, the excess goes to waste. The excess can not be reused due to concerns of contamination from patient connected catheters or other devices. This device is designed to prevent contamination by providing a design that allows contrast or other liquid media to flow to the patient while preventing the reverse flow of contaminants from the patient back to the source. This significantly minimized medical waste by allowing more efficient use of liquids like contrast while preventing the spread of disease from patient to patient.

This disclosure is written to describe a system that can prevent waste of contrast material used during angiographic procedures, but the technology described can be applied to any liquid pharmaceutical or other high value liquid being dispensed from a primary container to a secondary container. As can be seen, there is a need for a device to allow save use of unused fluids for subsequent patients or other uses.

### BRIEF SUMMARY OF THE INVENTION

One aspect of the present invention is a device to be attached to a contrast bottle comprising: a dosing unit having a bottle spike, a multi-function valve, a dispensing tube, and guides; a receiving tube; and a docking station; wherein the dosing unit is attached to a contrast bottle with the bottle spike, the two are added as a unit to the docking station, which holds these firmly in place in a vertical orientation, a receiving tube is advanced through the guides of the dosing unit thus maintaining vertical orientation and also preventing contamination, and the multi-function valve in the dosing unit is opened allowing contrast to flow into the receiving tube (receiver).

The device can be designed to operate without being connected to an internal or external power supply making it portable and improving reliability by eliminating the need for an external power source.

These and other features, aspects and advantages of the present invention will become better understood with reference to the following drawings, description and claims.

### BRIEF DESCRIPTION OF THE DRAWINGS

FIG. 1 depicts a side view of the stacked arrangement embodiment of the device;

FIG. 2 depicts a side view of the side-by-side arrangement embodiment of the device;

FIG. 3 depicts a side view of an exemplary embodiment of the receiver tube assembly splash and tipping shields with the receiver tube in vertical or horizontal positions;

FIG. 4 depicts a side view of an exemplary embodiment of the empty port plug and fluid level indicator with the valve in the open or the closed position;

FIG. 5 depicts a side view of an exemplary embodiment of the check valve;

FIG. 6 depicts a side view of an exemplary embodiment of the device assembled with components typically used for a catheterization procedure.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features. However, any single inventive feature may not address many of the problems discussed above or may only address one of the problems discussed above. Further, one or more of the problems discussed above may not be fully addressed by any of the features described below.

Broadly, embodiments of the present invention generally provide a device to allow the unused portion of contrast or other medical fluid to be used with the next patient. By using a large source of contrast (1), such as a bottle, with a docking unit (2), controlled amounts of contrast may be added to a patient line (3) and then the extra contrast can be used for the next patient with the use of a fresh receiver (4).

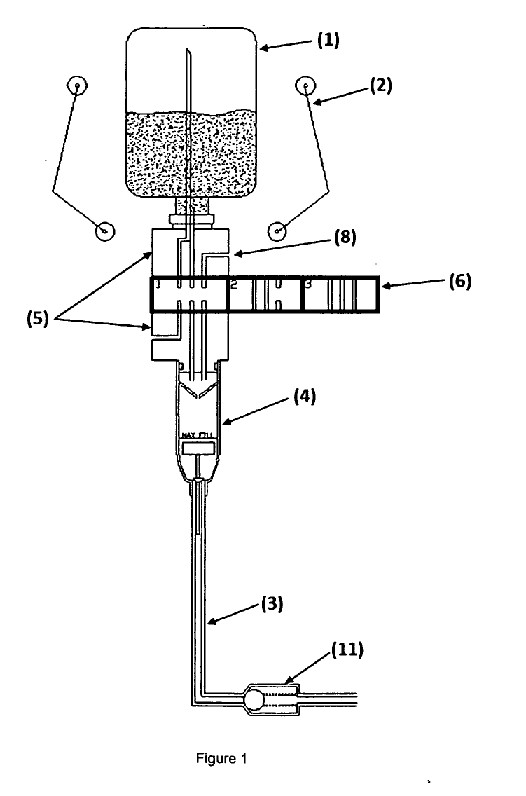

The basic operating principal of the fluid saver is the fluid is metered from the sterile jar (1) into the receiving tube or receiver (4) (see FIG. 1). Metering is achieved by sealing between the contrast jar, doser (5) with multi function valve assembly (6), and receiver. In the stacked orientation air is purged from the receiver such that the fluid in the jar is supported by the pressure in the air above the fluid in the receiver. As fluid is delivered to the patient the level in the receiver drops causing the air pressure to drop and allowing fluid to flow into the receiver until the pressure due to the gravity on the fluid and the air pressure in the receiver balance one another and flow stop.

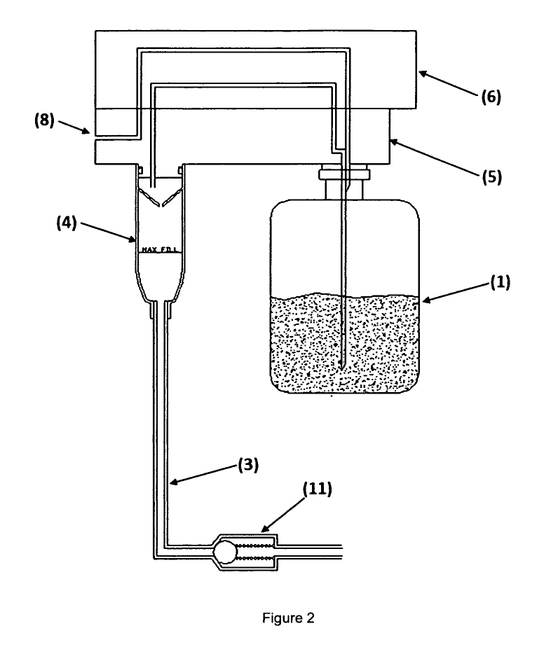

In an alternative embodiment depicted in FIG. 2, a side by side design (7) achieves the same basic function by inverting the bottle and drawing the fluid from the bottle using vacuum. The level of the vacuum is balanced by the weight of the column of fluid being sucked out of the bottle. When the pressure differential on the build caused by the vacuum and the weight of the fluid balance the flow stops. When fluid is delivered to the patient the vacuum increases and flow begins again until the pressure balances again.

The contrast saver comprises four components: the jar of contrast itself (1), the fluid retainers, doser (5), and receiver (4) and patient connected tube (3) (see FIGS. 1,2 and 6).

The doser is attached to a contrast bottle. The doser has an air vent tube (8) that can be pushed through the rubber stopper of the contrast bottle, as well as a dispensing tube. The doser has a mechanically actuated multi-function valve that opens when the receiving docking tube unit (receiver) is attached. The doser has guides that ensure coaxial and sterile docking with the receiver.

The docking station has a built in rest for the contrast bottle, as well as a secure mechanical attachment consisting of rollers, levers, springs and hinges as needed to secure the bottle. The docking station also has a system to secure it to an IV pole or other vertical mount comprising a hook, knob, screw and plate. The purpose of the station is to keep the bottle upright and doser upright. This aids in maintaining sterility. The station can also have a UV lamp array that further maintains sterility which encloses the dosing unit and receiver.

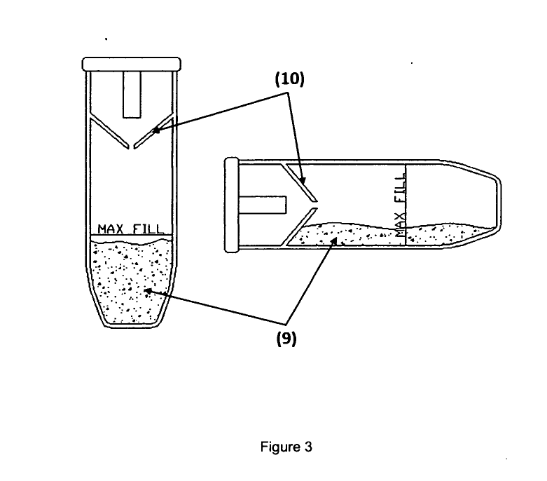

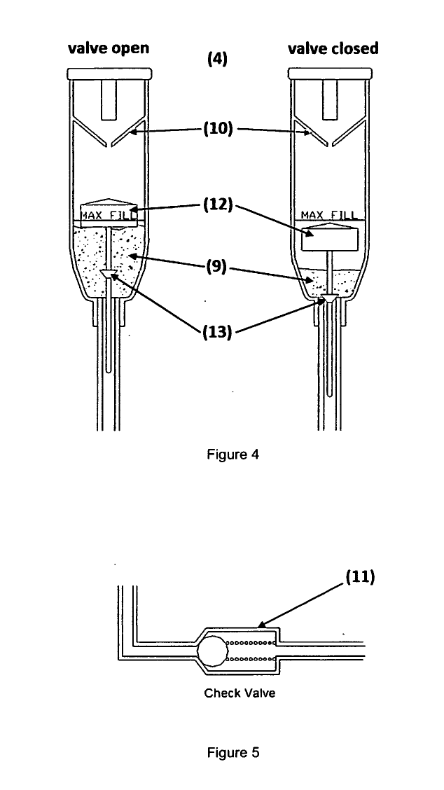

The receiver has a small reservoir (9) followed by patient tubing to allow contrast material to be used by the angiographer. The receiver trips the mechanical valve when attached to the doser (FIGS. 3 and 4).

The receiving tube is designed to maintain the sterility of the contrast in the supply by separating the patient connected tube from the supply by an air gap. The shape, length and diameter of the receiving tube can be adjusted to optimize performance. The sides of the receiver can be rigid or flexible. Flexible sides facilitate setting the fluid level allowing the user to squeeze the receiver to eject air as needed. In addition the receiver can have additional features to prevent the contamination of the supply that would occur from tipping, bumping, shaking, or vibration that could possibly occur in a busy hospital setting. FIG. 3 shows one embodiment of a splash preventing shield (10) that would deflect any patient connected fluid that splashes back down toward the patent connected fluid. In the extreme case when the fluid saver is completely tipped on its side these same features in the receiver act to maintain separation between the contrast supply and the patient connected fluid.

Also the connection between the receiver and the doser can be through many mechanical means including threads, twist locks, latches, slip fitted parts, and the like, and the seal between the two done with several possible sealing methods including a gasket, o-ring, luer fitting, and the like provided the receiver can be easily removed and a seal with the doser is maintained while the device is in use.

In one embodiment of the device there is a component within the receiver that assures there is a minimum fluid level in the receiver. The main reason to have this is to prevent air from getting into the patient line when the main contrast supply runs empty. When there is the correct fluid level in the receiver the empty port plug with its attached float (12) raises with the fluid level (see FIG. 4). Connected to the float is a rod that has a feature that acts as a valve (13) allowing fluid to flow into the patient connected tube. If the fluid level in the receiver drops below a minimum required to operate safely, the flow to the patient connected tube is blocked. If the contrast bottle is empty it can be changed for a new bottle, fluid can be added to the drip chamber, and the procedure can continue.

Check valves (11) are a safety feature that can be incorporated into either the patient connected tube or doser to assure fluid always flow in the direction of the patient (see FIG. 5). As a safety feature it is best to prevent retrograde flow in the system. Fluid should generally only flow from the sterile supply to the patient and not in the reverse direction.

The basic design shown can be extended to incorporate a second bottle so that when the first bottle runs empty the device can switch to run off a second bottle so that the first bottle can be changed and the device can continue to switch between two or more containers. This can be done with a sensor on the fluid level that causes a solenoid valve or other valve mechanism to switch bottles.

Another item is to incorporate a feature into the doser or multi function valve is to add a bottle lock to the valve that connects the patient connected supply bottle to the fluid saver. The lock disengages with the valve in the off position and engages for all other valve positions. The lock can physically engage the bottle or lock the retainers the hold the bottle. There are multiple benefits to this feature. It prevents the bottle from being removed or getting knocked off inadvertently, but also assures the valve is returned to the proper off position before a bottle is removed or replaced.

As an additional measure to assure sterility is maintained, an ultraviolet (UV) source can be added to the device at either the doser or receiver to intermittently or continuously irradiate the fluid to help maintain the sterile environment within the system.

An optical fluid level sensor can be added to the system to alert the user that the bottle supply is beginning to run low or is already empty. The detector can be configured to a light-emitting diode (LED) or other visual or audible indication that alerts the user that fluid is running low. LED's can be used to show the device status using color changes or flashing to communicate the status of the device.

The contract saver system can be designed to be pole mounted like many hospital devices to facilitate positioning or transport, or can also be designed to operate on a table top or other means of support.

Lastly, the design is not limited by the packaging of the fluid media. The device can be configured to use various types of contract packaging including a bag or other container.

In one embodiment the device works by allowing small doses of contrast material to be used at any one time. When the next patient arrives, the doser and docking station remain in place, while the receiver is changed for each new patient. The doser and receiver form an airtight seal, so the amount of contrast used will cause the dosing unit to refill the receiver reservoir. The doser has vertical guides in a circular array so that only the receiving tube may be advanced and attached, aiding sterility. The docking station provides a firm platform for the entire unit, preventing loss of sterility as well as providing UV irradiation to further aid in maintaining sterility. The multifunction valve is important in initially setting up the proper fluid level in the receiver, as well as adjusting the fluid level as needed during the procedure, to maintain an optimal fluid level in the receiver. In addition, at the anticipated end of the procedure, the valve can be set to “drain” position (not pictured, see description below), which will empty the receiver of all remaining contrast and lock the supply bottle. In this way, additional contrast material may be saved rather than discarded in the receiver at the end of the procedure.

In order to use the device, a contrast bottle is perforated with the bottle spike of the dosing unit. The two are added (as a unit) to the docking station, which holds these firmly in place, and in a vertical orientation. A receiving unit is then advanced through the vertical guides of the doser, maintaining vertical orientation and also preventing contamination. When the airtight seal is ensured, the valve in the doser is opened allowing contrast to flow into the receiver. The multi-function valve is operated to adjust the fluid level in the receiver. When contrast is withdrawn from the receiver with the multi-function valve in the operating position, the level in the receiver will fall, and more contrast will flow from the doser into the receiver through the contrast supply tube in the doser. The three operating positions for the multifunction valve are “fill”, “operate”, and “off”. An additional “drain” position is feasible using the multifunction valve, (not shown) but may be useful for lowering the level of contrast in the case of overfilling.

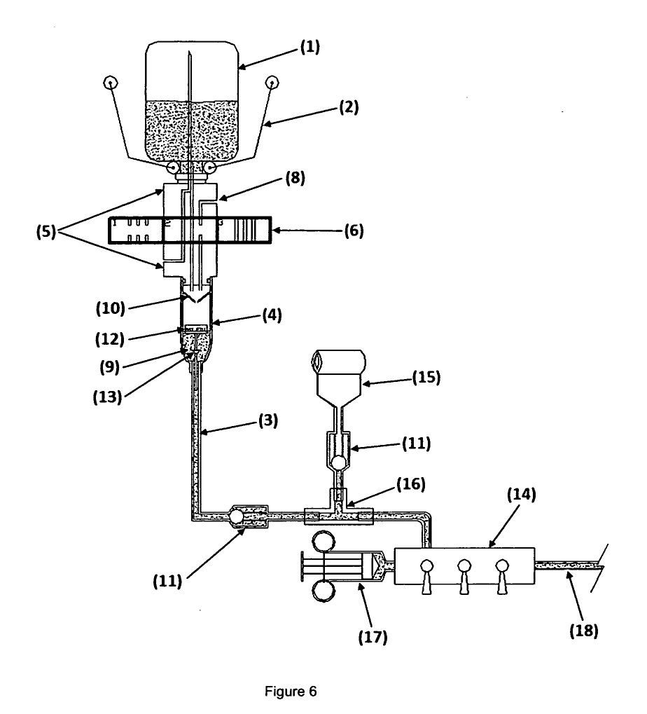

The device set up for use is shown in FIG. 6. Included are a manifold (14), dump bag (15), fittings (16), syringe (17), and catheter (18). These components are depicted to show how the fluid saver can be used with the materials that are typically used for a catheterization procedure. Use of the fluid saver does not require any change to standard materials or surgical procedures. The only change is the advantage that the contrast bottle does not need to be changed with each patient allowing all the contrast to be used resulting in significant cost savings.

The invention may be made with routine manufacturing techniques found in the medical devices/technology field, and could easily be manufactured by any one of a number of medical devices companies. The doser and receiving units may be made of plastic, and the docking station may be made of plastic or aluminum, or any other suitable material.

The approach described herein is described based on the use of contrast medium. This value of the system is to prevent waste of liquid materials that would otherwise not be allowed to be used based on sterility, mixing, or other contamination concerns. But the same approach can be used for other medications any time drug waste is an issue. Also the use of the device is not limited to medical applications. The approach described in this system is applicable any time contamination between a delivered liquid and its supply must be prevented. The device described can be used in any other field or technology where fluids are being used and contamination free dispensing is needed.

It should be understood, of course, that the foregoing relates to exemplary embodiments of the invention and that modifications may be made without departing from the spirit and scope of the invention as set forth in the following claims.

1. A device designed to deliver fluid for a supply to a connected point of use without allowing contamination from the point of use back to the supply. Contamination can be considered any retrograde flow of the liquid being delivered or other material introduced by the point of use connected part. The device comprising any or all of the following components:
a dosing unit;
a multi-function valve that can include a drain position as needed;
a dispensing tube;
a receiving tube; and
a docking station.

2. The device in claim 1 which incorporates a float valve or other similar mechanism to assure a minimum amount of liquid is always available in a reservoir in the point of use connected tubing.

3. The device in claim 1 which incorporates a mechanism like a splash shield to prevent contamination back to the supply when the mechanism experiences shaking and vibration from handling or transport. The splash shield also prevents contamination back to the supply is the device is tipped off axis.

4. The device in claim 1 which can be mounted on a tabletop, free standing, clamped to another apparatus, mounted to a portable stand or pole, or integrated into another larger fluid handling system.

5. The device in claim 1 that is designed to accommodate a backup supply where the multifunction valve can easily switch from one supply to another.

6. The device in claim 1 that is intended to perform its function without the use of an internal or external power supply.

7. The device in claim 1 which utilizes a check valve to assure one way flow to the point of use connected part.

8. The device in claim 1 which incorporates a sensor to monitor fluid level to alert the user the fluid supply is nearly exhausted.

9. The device in claim 1 which incorporates a optical, thermal, filter, or other means to prevent contamination of the supply from the point of use connected part.

10. The device in claim 1 which can be connected to numerous supply containers including rigid and flexible sided packages.

11. The device in claim 1 with a receiver that can be rigid or flexible to allow the user to squeeze the receiver forcing air through the system which allows control of the volume of fluid maintained in the receiver.

12. The device in claim 1 which allows rapid and secure clamping of a defined supply container through any combination of springs, rollers, bands, Velcro, or other secure and easily overridden latch.

13. A multifunction valve that is keyed to the presence of the supply container so the valve must be switched to a prescribed position before the supply container can be removed or replaced.

14. The device in claim 1 which can be used to deliver pharmaceuticals to a patient while maintaining sterility of the supply container.

15. A device to be attached to a contrast bottle comprising any or all of the following components:
a dosing unit having a bottle spike, a multi-function valve, a dispensing tube, and guides;
a receiving tube; and
a docking station;
wherein the dosing unit is attached to a contrast bottle with the bottle spike, the two are added as a unit to the docking station, which holds these firmly in place, a receiving tube attaches to the dosing unit forming a seal allowing fluid to be delivered to the receiver while preventing contamination between the receiving tube and contrast bottle, and the multi-function valve in the dosing unit is opened allowing contrast to flow into the receiver in a controlled manner.
